(19) United States
(12) Patent Application Publication
(10) Pub. No.: US 2023/0284948 A1
(43) Pub. Date: Sep. 14, 2023

(54) TEST PROTOCOL FOR DETECTING SIGNIFICANT PSYCHOPHYSIOLOGICAL RESPONSE

(71) Applicant: PANGEA LABS LTD., Herzliya (IL)

(72) Inventors: Ekaterina KON, Ashdod (IL); Elliot SPRECHER, Tel Aviv (IL); Yuval ODED, Tel Aviv (IL); Dmitry GOLDENBERG, Ashdod (IL)

(21) Appl. No.: 18/015,235

(22) PCT Filed: Jul. 7, 2020
(86) PCT No.: PCT/IL2020/050760

§ 371 Date: Jan. 9, 2023

(51) Int. Cl. A61B 5/16; A61B 5/00; G16H 10/20; G16H 50/20

(52) U.S. Cl. CPC A61B 5/165; A61B 5/7267; G16H 10/20; G16H 50/20

(57) ABSTRACT

A method comprising receiving, as input, physiological parameters data measured in a human subject in response to an administered test question protocol comprising (a) a plurality of test question segments, each comprising at least one test question, and (b) a recovery period following each of the test question segments; determining a stress signal associated with the test question protocol, based, at least in part, on one or more states of stress detected in the physiological parameters data; temporally associating values of the stress signal with the plurality of test question segments and the recovery periods; and calculating, for at least some of the test question segments, a segment psychophysiological response score associated with the responses by the subject, based on an analysis of the temporally associated values of the stress signal.

### CROSS REFERENCE TO RELATED APPLICATIONS

This application claims priority from U.S. Patent Application No. 62/871,148, filed on Jul. 7, 2019, entitled “TEST PROTOCOL FOR DETECTING SIGNIFICANT PSYCHOPHYSIOLOGICAL RESPONSE,” the contents of which are incorporated by reference herein in their entirety.

### BACKGROUND

The invention relates to the field of computerized psychophysiological response analysis.

Human psychophysiological behavior can be described as a combination of different physiological stress types. Stress, in turn, may be described as a physiological response to internal or external stimulation, and can be observed in physiological indicators. When external or internal stimulations are created, they may cause the activation of the hypothalamus brain system to activate different processes, which influence the autonomic nervous system and sympathetic and parasympathetic systems, which ultimately control the physiological systems of the human body.

Psychophysiological testing, like all diagnostic activities, involves using specific observations to ascertain underlying, less readily observable, characteristics. For example, polygraph testing is used as a direct measure of physiological responses and as an indirect indicator of whether an examinee is telling the truth, based on the belief that deceptive answers will produce physiological responses that can be differentiated from those associated with non-deceptive answers.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive, as input, physiological parameters data measured in a human subject in response to an administered test question protocol comprising (a) a plurality of test question segments, each comprising at least one test question, and (b) a recovery period following each of the test question segments, determine a stress signal associated with the test question protocol, based, at least in part, on one or more states of stress detected in the physiological parameters data, temporally associate values of the stress signal with the plurality of test question segments and the recovery periods, and calculate, for at least some of the test question segments, a segment psychophysiological response score associated with the responses by the subject, based on an analysis of the temporally associated values of the stress signal.

There is also provided, in an embodiment, a method comprising: receiving, as input, physiological parameters data measured in a human subject in response to an administered test question protocol comprising (a) a plurality of test question segments, each comprising at least one test question, and (b) a recovery period following each of the test question segments, determining a stress signal associated with the test question protocol, based, at least in part, on one or more states of stress detected in the physiological parameters data, temporally associating values of the stress signal with the plurality of test question segments and the recovery periods, and calculating, for at least some of the test question segments, a segment psychophysiological response score associated with the responses by the subject, based on an analysis of the temporally associated values of the stress signal.

There is further provided, in an embodiment, a computer program product comprising a non-transitory computer-readable storage medium having program code embodied therewith, the program code executable by at least one hardware processor to: receive, as input, physiological parameters data measured in a human subject in response to an administered test question protocol comprising (a) a plurality of test question segments, each comprising at least one test question, and (b) a recovery period following each of the test question segments; determine a stress signal associated with the test question protocol, based, at least in part, on one or more states of stress detected in the physiological parameters data; temporally associate values of the stress signal with the plurality of test question segments and the recovery periods; and calculate, for at least some of the test question segments, a segment psychophysiological response score associated with the responses by the subject, based on an analysis of the temporally associated values of the stress signal.

In some embodiments, the segment psychophysiological response is a significant responses (SR).

In some embodiments, the test question protocol starts with a baseline period comprising instructing the subject to perform a plurality of undemanding cognitive tasks.

In some embodiments, the analysis comprises calculating at least one of: (i) a test question protocol stress signal global baseline associated with the subject, based, at least in part, on the values of the stress signal during the baseline period; and (ii) with respect to each test question segment, a stress signal segment baseline, based, at least in part, on the global baseline and a value of the stress signal during a the recovery period immediately preceding the test question segment.

In some embodiments, the analysis comprises, with respect to a test question segment of the test question segments, calculating at least one of: (i) reaction times associated with each of the responses to each of the test questions; (ii) an intensity value of the stress signal associated with the test question segment, relative to the test question segment baseline; and (iii) an intensity and variability values of the stress signal during a the recovery period immediately following the test question segment, relative to the global baseline.

In some embodiments, the segment psychophysiological response score is based, at least in part, on the calculating.

In some embodiments, the analysis comprises detecting one or more reaction sections in the stress signal, based, at least in part, on an increase in the value of the stress signal relative to a local minimum. In some embodiments, the analysis further comprises calculating an area under a curve associated with each of the reaction sections. In some embodiments, the analysis further comprises calculating a test question protocol stress signal global baseline associated with the subject, based, at least in part, on an (i) average of all of the areas under the curve associated with each of the reaction sections, and (ii) a variability of all of the areas under the curve associated with each of the reaction.

In some embodiments, the segment psychophysiological response score is based, at least in part, on a sum of all of the areas under the curve associated with each of the reaction sections, associated with the respective test question segment, relative to the global baseline.

In some embodiments, the segment psychophysiological response score is based, at least in part, on a reaction score associated with the test question segment, equal to a duration of the reaction section relative to a standard reaction duration, multiplied by an intensity value of the stress signal during the reaction section.

In some embodiments, the program instructions are further executable to calculate, and the method further comprises calculating, a test question protocol psychophysiological response score, based, at least in part, on a weighted sum of all of the segment psychophysiological response scores.

In some embodiments, the weighting is based on one of: score severity and test question segment importance ranking.

In some embodiments, the states of stress are selected from the group consisting of: neutral stress, cognitive stress, positive emotional stress, and negative emotional stress.

In some embodiments, the stress signal is calculated, at least in part, by combining at least one of the detected cognitive stress, positive emotional stress, and negative emotional stress.

In some embodiments, the physiological parameters data are acquired using one or more of: an imaging device, an infrared (IR) sensor; a hyperspectral imaging device; a skin surface temperature sensor; a skin conductance sensor; a respiration sensor; a peripheral capillary oxygen saturation (SpO2) sensor; an electrocardiograph (ECG) sensor; a blood volume pulse (BVP) sensor; a heart rate sensor; a surface electromyography (EMG) sensor; an electroencephalograph (EEG) acquisition sensor; a joint bend sensor; and a muscle activity sensor.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a block diagram of a system for training a machine learning classifier to detect a state of stress in a human subject, according to an embodiment;

FIG. 2 is a flowchart of a data analysis process of an exemplary system for detecting a psychophysiological response in a subject, according to an embodiment;

FIG. 3 is a block diagram schematically illustrating an exemplary psycho-physiological stress test protocol, according to an embodiment; and

FIG. 4 is a block diagram schematically illustrating an exemplary psycho-physiological response questionnaire, according to an embodiment;

FIG. 5A is a schematic illustration of an exemplary test protocol according to an embodiment;

FIG. 5B is a flowchart detailing the functional steps in administering an exemplary psycho-physiological test protocol configured for exposing stimulation series segments to a participant, according to an embodiment;

FIG. 6 shows a stimulation series subset metadata vector, utilize for record the flow of a stimulation series subset, according to an embodiment;

FIG. 7 is a flowchart of the functional steps in an algorithm of the present disclosure, according to an embodiment; and

FIG. 8 is an illustration of reaction area calculation, according to an embodiment.

### DETAILED DESCRIPTION

Disclosed herein are a method, system, and computer program product for detecting a state of psychophysiological response in a subject, based, at least in part, on detecting a combination of one or more stress signals in physiological parameters data acquired from a subject.

In some embodiments, psychophysiological response may be defined as measurable physiological responses in a subject, associated with responding to one or a series of relevant test questions. psychophysiological response may include, e.g., significant response (SR), which may be defined as a consistent, significant, and timely. For example, a significant response detected in a subject in response to one or a series of test questions, may indicate an intention on part of the subject to provide a false or deceptive answer to the test questions.

In some embodiments, a dedicated global stress analysis algorithm of the present invention may be configured for detecting SR based, at least in part, on an input comprising various stress signals detected in a subject. In some embodiments, the various stress signals may be detected using a machine learning classifier configured for detecting individual categories of stress in a subject, including, but not limited to:
Neutral stress: A neutral state in which stimulations do not induce cognitive or emotional responses.
Cognitive stress: Stress associated with cognitive processes, e.g., when a subject is asked to perform a cognitive task, such as to solve a mathematical problem.
Positive emotional stress: Stress associated with positive emotional responses, e.g., when a subject is exposed to images inducing positive feelings, such as happiness, exhilaration, delight, etc.
Negative emotional stress: Stress associated with negative emotional responses, e.g., when a subject is exposed to images inducing fear, anxiety, distress, anger, etc.

In some embodiments, a ‘global stress’ signal may be further determined, wherein global stress may be defined as an aggregate value of the one or more individual stress states in the subject. In some embodiments, a global stress value in a subject may be determined by summing the values of detected cognitive and/or emotional stress in the subject. In some variations, the aggregating may be based on a specified ratio between the individual stress categories.

In some embodiments, the present invention may further provide for detection of an additional state of stress known as ‘continuous expectation stress’ in a subject, wherein continuous expectation stress may be defined as a state of suspenseful anticipation, e.g., when a subject is expecting an imminent significant or consequential event. In some embodiments, the detection may be based on at least one of (i) a specified combination of one or more of the other stress categories detected in the subject, (ii) a machine learning classifier trained to detect continuous expectation stress in other stress signals, and (iii) a machine learning classifier trained to detect continuous expectation stress in physiological parameters acquired from a test subject.

In some embodiments, the machine learning classifier may be trained on a training set comprising physiological parameters data acquired from a plurality of test participants, wherein the physiological parameters data is associated with various categories of stress induced in each test participant. In some embodiments, the physiological parameters data set on which the training set is based may be acquired by a suitable acquisition system, during the course of administering one or more psycho-physiological test protocols to the test participants, wherein the test protocols may be configured for inducing one or more of the individual categories of stress. In some embodiments, the detection of the categories of stress may be based, at least in part, on determining a typical range of physiological parameters associated with each such category of stress.

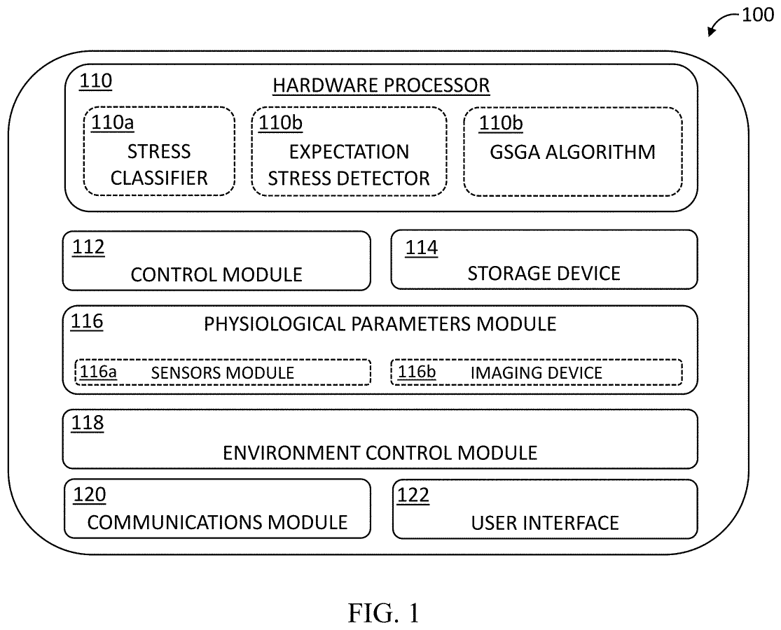

FIG. 1 is a block diagram of an exemplary system 100 according to an embodiment of the present invention. System 100 as described herein is only an exemplary embodiment of the present invention, and in practice may have more or fewer components than shown, may combine two or more of the components, or a may have a different configuration or arrangement of the components. The various components of system 100 may be implemented in hardware, software or a combination of both hardware and software. In various embodiments, system 100 may comprise a dedicated hardware device, or may form an addition to or extension of an existing device.

In some embodiments, system 100 may comprise a hardware processor 110 having a stress classifier 110a, an expectation stress detector 110b, and a global stress analysis algorithm module 110c; a control module 112, and a non-volatile memory storage device 114; a physiological parameters module 116 having, e.g., a sensors module 116a and an imaging device 116b; environment control module 118; communications module 120; and user interface 122.

System 100 may store in storage device 114 software instructions or components configured to operate a processing unit (also “hardware processor,” “CPU,” or simply “processor), such as hardware processor 110. In some embodiments, the software components may include an operating system, including various software components and/or drivers for controlling and managing general system tasks (e.g., memory management, storage device control, power management, etc.) and facilitating communication between various hardware and software components.

In some embodiments, physiological parameters module 112 may be configured for acquiring a plurality of physiological parameters data from human subjects. In some embodiments, sensors module 116a may comprise at least some of:
Infrared (IR) sensor for measuring bodily temperature emissions;
skin surface temperature sensor;
skin conductance sensor, e.g., a galvanic skin response (GSR) sensor;
respiration sensor;
peripheral capillary oxygen saturation (SpO2) sensor;
electrocardiograph (ECG) sensor;
blood volume pulse (BVP), also known as photoplethysmography (PPG), sensor;
heart rate sensor;
surface electromyography (EMG) sensor;
electroencephalograph (EEG) acquisition sensor;
bend sensor, to be placed on fingers and wrists to monitor joint motion; and
sensors for detecting muscle activity in various areas of the body.

In some embodiments, imaging device 116b may comprise any device that captures images and represents them as data. Imaging devices 116b may be optic-based, but may also include depth sensors, radio frequency imaging, ultrasound imaging, infrared imaging, and the like. In some embodiments, imaging device 116b may be a Kinect or a similar motion sensing device, capable of, e.g., IR imaging. In some embodiments, imaging device 116b may be configured to detect RGB (red-green-blue) spectral data. In other embodiments, imaging device 116b may be configured to detect at least one of monochrome, ultraviolet (UV), near infrared (NIR), and short-wave infrared (SWIR) spectral data.

In some embodiments, environment control module 118 comprises a plurality of sensors configured for monitoring environmental conditions at a testing site. Such sensors may include, e.g., lighting and temperature conditions, to ensure consistency in environmental conditions among multiple test subjects. For example, environment control module 118 may be configured to monitor an optimal ambient lighting in the test environment between 1500-3000 lux units, e.g., 2500. In some embodiments, environment control module 118 may be configured to monitor an optimal ambient temperature in the test environment, e.g., between 22-24° C.

In some embodiments, communications module 120 may be configured for connecting system 100 to a network, such as the Internet, a local area network, a wide area network and/or a wireless network. Communications module 120 facilitates communications with other devices over one or more external ports, and also includes various software components for handling data received by system 100. In some embodiments, a user interface 122 comprises one or more of a control panel for controlling system 100, display monitor, and a speaker for providing audio feedback. In some embodiments, system 100 includes one or more user input control devices, such as a physical or virtual joystick, mouse, and/or click wheel. In other variations, system 100 comprises one or more of a peripherals interface, RF circuitry, audio circuitry, a microphone, an input/output (I/O) subsystem, other input or control devices, optical or other sensors, and an external port. Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, control module 112 is configured for integrating, centralize and synchronize control of the various modules of system 100.

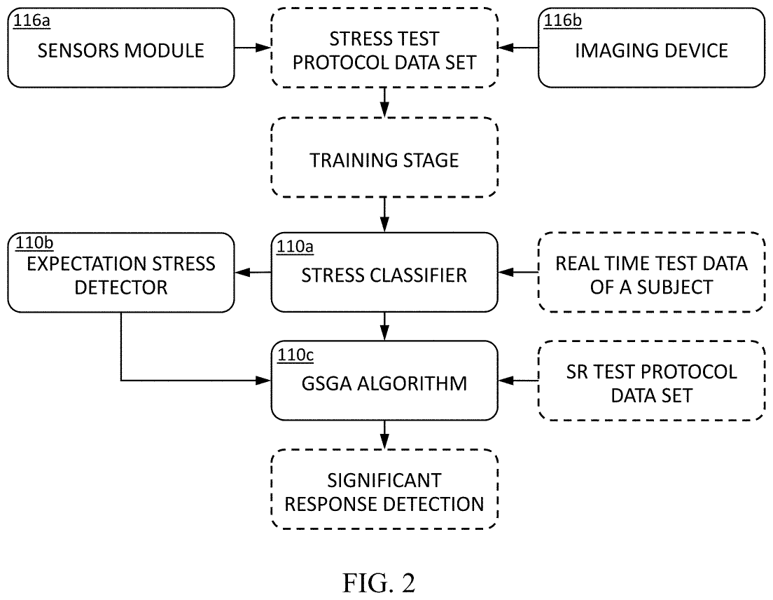

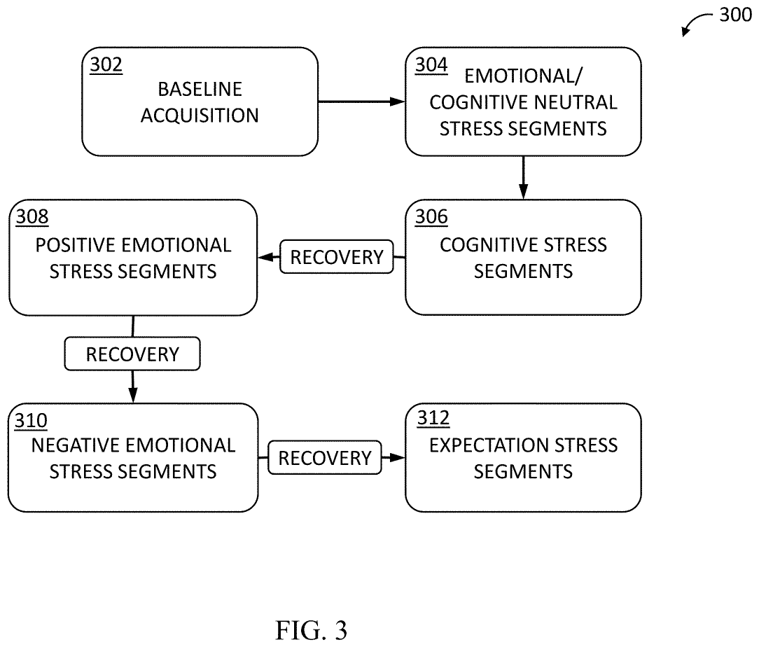

An overview of the functional steps in a process for detecting SR in a data set of physiological parameters acquired from a subject, using a system such as system 100, will be provided with reference to the block diagram in FIG. 2. In some embodiments, at a training stage, system 100 may be configured for acquiring a data set comprising physiological parameters from a plurality of human test participants, wherein the physiological parameters are being acquired in the course of administering one or more psycho-physiological test protocols to each of the participants (as will be further described below with reference to FIG. 3). System 100 may then use the data set to generate a training set for training stress classifier 110a to classify one or more categories of stress. In some embodiments, a trained stress classifier 110a may be configured for determining a global stress signal based, at least in part, on the classification of stress categories in physiological parameters data.

In some embodiments, expectation stress detector 110b may be configured for receiving the output from stress classifier 110a to further detect continuous expectation stress in the signals. In other embodiments, expectation stress detector 110b may be configured for detecting continuous expectation stress based on, e.g., one of a machine learning classifier trained to detect continuous expectation stress in other stress signals, and a machine learning classifier trained to detect continuous expectation stress in physiological parameters acquired from a test subject.

In some embodiments, a trained stress classifier 110a may then receive as input physiological parameters data acquired from a test subject in answering one or more relevant questions, and process the data to output stress signals, e.g., a global stress signal. In some embodiments, global stress analysis algorithm 110c may be configured for detecting SR based, at least in part, on processing (i) a global stress signal received from stress classifier 110a, and/or (ii) a continuous expectation stress signal received from expectation stress detector 110b. In other embodiments, global stress analysis 110c may be configured for detecting SR based on additional and/or alternative sources of input including, but not limited to, additional and/or other stress signals, and/or raw physiological parameters data acquired from a subject.

In some embodiments, a data set generated by system 100 for the purpose of generating the training set for stress classifier 110a may be based on physiological parameters data acquired from between 50 and 450 test participants, e.g., 150 test participants. In other embodiments, the number of participants may be smaller or greater. In some embodiments, all participants may undergo identical test protocols. In other embodiments, sub-groups of test participants selected at random from a pool of potential participants may be administered different versions of the test protocol.

In some embodiments, a test protocol may be administered by a specialist, be a computer-based test, or combine both approaches. In cases where a test protocol may be administered by a specialist, test participants may be seated near the specialist so as to induce a degree of psychological pressure in the participant, however, in such a way that test participant and specialist do not directly face each other, to avoid any undue influence of the specialist on the participant. In addition, participants may be instructed to sit upright, with both legs touching the ground, and to avoid, to the extent possible, body, head, and/or hand movements.

In some embodiments, test participants may be selected from a pool of potential participants comprising substantially similar numbers of adult men and women. In some embodiments, potential test participants may undergo a health and psychological screening, e.g., using a suitable questionnaire, to ensure that no test participant has a medical and/or mental condition which may prevent the participant from participating in the test, adversely affect test results, and/or manifest in adverse side effects for the participant. For example, test participants may be screened to ensure to no test participant takes medications which may affect test results, and/or currently or generally suffers adverse health conditions, such as cardiac disease, high blood pressure, epilepsy, mental health issues, consumption of alcohol and/or drugs within the most recent 24 hours, and the like.

In some embodiments, physiological parameters module 116 may be configured for continuously acquiring and monitoring, during the course of administering the test protocols to participants, a plurality of physiological parameters from the participant. Such physiological parameters may include, but are not limited to, a video stream of the whole body, the face alone, and/or other body parts, of the participant, taken by imaging device 116b. In other embodiments, physiological parameters module 116 may be configured for taking measurements relating to bodily temperature; heart rate; heart rate variation (HRV); blood pressure; blood oxygen saturation; skin conductance; respiratory rate; eye blinking; pupil movement; ECG; EMG; EEG; PPG; finger/wrist bending; and/or muscle activity. Similarly, environment control module 118 may be configured for continuously monitoring ambient conditions during the course of administering the test protocol, including, but not limited to, ambient temperature and lighting.

In some embodiments, each psycho-physiological test protocol comprises a series of between 2 and 6 stages. During each of the stages, participants may be exposed to between 1 and 4 stimulation segments, each configured to induce one of the different categories of stress described above, including neutral emotional or cognitive stress, cognitive stress, positive emotional stress, negative emotional stress, and/or continuous expectation stress. In some embodiments, each test stage may last between 20 and 600 seconds. In some embodiments, all stages have an identical length, e.g., 360 seconds. In some embodiments, each segment within a stage may have a length of between 30 and 400 seconds. In some embodiments, test segments designed to induce continuous expectation stress may be configured for lasting at least 360 seconds, so permit the buildup of suspenseful anticipation.

In some embodiments, the various stages and/or individual segments within a stage may be interspersed with periods of break or recovery configured for unwinding a stress state induced by the previous stimulation. In some embodiments, each recovery segment may last, e.g., 120 seconds. In some embodiments, recovery segments may comprise exposing a participant to, e.g., relaxing or meditative background music, changing and/or floating geometric images, and/or simple non-taxing cognitive tasks. For example, because emotional stress stimulations may have a heightened and/or more lasting effect on participants, recovery segments following negative emotional stimulations may comprise simple cognitive tasks, such as a dots counting task, configured for neutralizing an emotional stress state in a participant.

FIG. 3 is a block diagram schematically illustrating an exemplary psycho-physiological test protocol 300 configured for inducing various categories of stress in a participant, according to an embodiment. In some embodiments, at a stage 302, system 100 may be configured for acquiring baseline physiological parameters of a test participant, in a state of rest where the participant may not be exposed to any stimulations.

At a stage 304, the participant may be exposed to one or more stimulations configured to induce a neutral emotional or cognitive state. For example, the participant may be exposed to one or more segments of relaxing or meditative background music, to induce a neutral emotional state. The participant may also be exposed to images incorporating, e.g., changing geometric or other shapes, to induce a neutral cognitive state.

Following the neutral stress stage, at a stage 306, the participant may be exposed to one or more cognitive stress segments, which may be interspersed with one or more recovery segments. For example, the participant may be exposed to a Stroop test asking the participant to name a font color of a printed word, where the word meaning and font color may or may not be incongruent (e.g., the word ‘Green’ may be written variously using a green or red font color). In other cases, a cognitive stimulation may comprise a mathematical problem task, a reading comprehension task, a ‘spot the difference’ image analysis task, a memory recollection task, and/or an anagram or letter-rearrangement task. In some cases, each cognitive task may be followed by a suitable recovery segment.

At a stage 308, the participant may then be exposed to one or more stimulation segments configured to induce a positive emotional response. For example, the participant may be exposed to one or more video segments designed to induce reactions of laughter, joy, happiness, and the like. Each positive emotional segment may be followed by a suitable recovery segment.

At a stage 310, the participant may be exposed to one or more stimulations configured to induce a negative emotional response. For example, the participant may be exposed to one or more video segments designed to induce reactions of fear, anger, distress, anxiety, and the like. Each negative emotional segment may be followed by a suitable recovery segment.

Finally, at a stage 312, the participant may be exposed to one or more stimulations configured to induce continuous expectation stress. For example, the participant may be exposed to one or more video segments showing a suspenseful scene from a thriller feature film. Each expectation segments may be also followed by a suitable recovery segments.

Exemplary test protocol 300 is only one possible such protocol. Alternative test protocols may include fewer or more stages, may arrange the stages in a different order, and/or may comprise a different number of stimulation and recovery segments in each stage. However, in some embodiments, test protocols of the present invention may be configured to place, e.g., a negative emotional segment after a positive emotional segment, because negative emotions may be lingering emotions which may affect subsequent segments.

In some embodiments, following the acquisition of the physiological parameters data set from a predetermined number of test participants using test protocol 300, stress classifier 110a may be configured for receiving the physiological parameters for each test participant from physiological parameters module 116. Stress classifier 110a may then be configured for temporally associating the physiological parameters data for each participant with the corresponding stimulation segments administered to the participant, using, e.g., appropriate time stamps. In some embodiments, the temporally-associated data set may comprise a training set for training stress classifier 110a to predict one or more of the constituent stress categories (i.e., neutral stress, cognitive stress, positive emotional stress, negative emotional stress, and/or continuous expectation stress). In some embodiments, stress classifier 110a may also be trained to detect a state of global stress in a human subject based, at least in part, on detecting a combination of one or more of the constituent stress categories in the set of measured physiological parameters.

In some embodiments, expectation stress detector 110b may be configured for detecting continuous expectation stress based, at least on part, on stress signals detected by stress classifier 110a. In other embodiments, continuous expectation stress may be detected based on training a machine learning classifier using a training set comprising raw physiological parameters data acquired from a plurality of human test participants, wherein such physiological parameters are acquired in the course of administering one or more psycho-physiological test protocols configured for inducing continuous expectation stress. In some embodiments, such machine learning classifier may be trained using data analysis windows of between 60-120 seconds each.

In some embodiments, global stress analysis algorithm 110c may be configured for detecting SR in a global stress signal received from stress classifier 110a, wherein the global stress signal is detected in physiological parameters data acquired from a subject in the course of answering an SR protocol. In some embodiments, the SR protocol may be configured for administering under similar environmental conditions to those described above with reference to test protocol 300 and FIG. 3.

In some embodiments, the SR protocol comprises one or more stages, e.g., 3 stages, wherein each stage comprises a set of between 5 and 10 SR ‘triggers.’ In other embodiments, the triggers may comprise simple questions. In other embodiments, the triggers may be additional and/or other verbal, audio and/or visual stimulations. In the case of questions, each set may include, e.g., 7 identical questions comprising, e.g., 5 ‘relevant’ questions (i.e., questions related to an event that is germane to the participant) and 2 ‘non-relevant’ questions. Each stage may repeat the same set of question in a different order. In some embodiments, the subject is afforded exactly 20 seconds to answer each question. Following each set of question, the participant may get 30 seconds of rest. The exemplary SR protocol is only one possible such protocol. Alternative test protocols may include fewer or more trigger sets, may arrange the sets in a different order, and/or may comprise a different number of triggers.

In some embodiments, a system, such as system 100 in FIG. 1, may be configured for continuously acquiring and monitoring, during the course of administering the SR protocol to a subject, a plurality of physiological parameters from the participants similar to those described with reference to test protocol 300 above. Such physiological parameters may include, but are not limited to, a video stream of the whole body, the face, and/or other body parts, of the participants; bodily temperature; heart rate; blood pressure; skin conductance; respiratory rate; blood oxygen saturation; ECG; EMG; EEG; finger/wrist bending; eye blinking; and/or muscle flexion. Similarly, environment control module 118 may be configured for continuously morning ambient conditions, including, but not limited to, ambient temperature and lighting.

In some embodiments, global stress analysis algorithm 110c may be configured for analyzing a global stress signal received from stress classifier 110a based on segmenting the signal into a plurality of analysis windows. In some embodiments, global stress analysis algorithm 110c may be configured for defining analysis windows based on time stamps, such that each window corresponds to a single trigger in the SR protocol. In some embodiments, each such analysis window may start at the start-time of the corresponding trigger, and end, e.g., between 1-5 seconds before the end of the trigger. In other embodiments, analysis windows may be defined in a variety of ways, including, but not limited to, with respect to analysis window length, number of triggers covered within a window, start/end times of each window, and/or sections of overlap between consecutive windows.

In some embodiments, global stress analysis algorithm 110c may be configured for calculating, with respect to each analysis window, an SR score based, at least in part, on the measured global stress signal in each analysis window. The SR score may be based, at least in part, on an integral of the global stress signal taken over the analysis window, relative to a baseline value. In some embodiments, global stress analysis algorithm 110c may be configured for calculating a combined SR score for each identical question in the 3 stages of the SR protocol, e.g., based on a mean score of the 3 appearances of the question. In some embodiments, global stress analysis algorithm 110c may then be configured for comparing the SR score of each question in the SR protocol to known detected responses acquired in a preliminary stage. global stress analysis algorithm 110c may then assign a nominal value of, e.g., between 1-5 to each SR score, wherein the nominal values represents the severity of the response relative to baseline response. In some embodiments, the baseline value may be measured as within 0-2 seconds from the start of the trigger. In some embodiments, global stress analysis algorithm 110c may be configured for calculating an absolute value of the change in global stress signal from the baseline, based on the observation that, in different subjects, SR may be expressed variously as increasing or decreasing (relief) trends of the global stress signal. In other embodiments, SR detection may be further based on additional and/or other statistical calculations with respect to each analysis window, or segments of an analysis window (dubbed epochs). Such statistical calculations may include, but are not limited to, mean values of the various epochs within an analysis window, standard deviation among epochs, and/or maximum value and minimum value within an analysis window.

In some embodiments, global stress analysis algorithm 110c may be further configured for analyzing the SR scores corresponding to the multiple triggers in an administered the SR protocol, wherein a trigger having the highest SR score (e.g., 6) may be designated as a significant trigger which may have occasioned a psychophysiological response in the subject. For example, in a case where the SR protocol may be a polygraph-type questionnaire, a highest scoring response may indicate an intention on part of the subject to provide a false or deceitful response.

In some embodiments, global stress analysis algorithm 110c may be further configured for receiving additional data from expectation stress detector 110b, to determine a state of continuous expectation stress with respect to one of the triggers in the SR protocol. A detected state of continuous expectation stress, which may be used in combination with the global stress analysis detailed above, may then provide a further indication that a particular trigger has occasioned a psychophysiological response in the subject, with an intention on part of the subject to provide a false or deceitful response. For example, a decline in a continuous expectation stress value may indicate an end of a suspenseful expectation period, in which the subject may have been anticipating an imminent significant trigger. Accordingly, a trigger accompanied by a decline in continuous expectation stress may indicate an SR event.

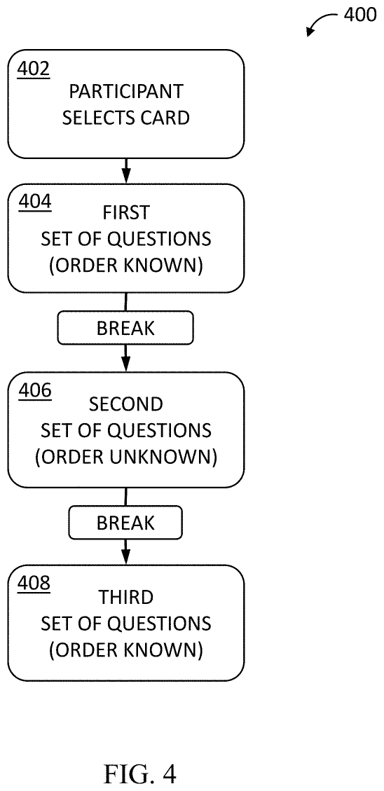

FIG. 4 is a block diagram of an exemplary SR protocol 400 which may be used in a verification stage for global stress analysis algorithm 110c. SR protocol 400 may be administered to a plurality of test participants, e.g. 30, to acquire a data set comprising physiological parameters. SR protocol 400 may comprise, e.g., 3 sets of 7 questions each. In some cases, a participant may be asked, e.g., to select a card from a deck of cards. Then, 5 of the questions in each set may be ‘relevant’ questions about the selected card, wherein the 5 relevant question are bookended by a first and last non-relevant question about other subject. In each set of questions, one question will be designated as a ‘key’ question for which the participant is directed to provide a false answer, and which may not be the first or last question in the set. In the first set, the participant may know the order of the questions. However, in the second set, the ‘key’ question will appear unexpectedly, so as to build an expectation stress response in the participant (after the first two sets, the participant will be able to deduce the order of questions in the third set).

Accordingly, at a stage 402 of SR protocol 400, a participant is directed to select a card from a deck comprising, e.g., 5 cards. At a stage 404, the participant may be directed to answer 7 questions whose order is known to the participant, wherein a ‘key’ relevant question in the set must be answered falsely. The participant is afforded exactly 20 seconds to answer each question. Following the first set of question, the participant may get 30 seconds of rest.

At a stage 406, the participant may be directed to answer the same 7 questions as in set one, however, in a different order, wherein the place of the ‘key’ question in the order is not known. At a stage 408, the participant may be directed to answer the same 7 questions again, in yet another order. Following the second set of question, the participant may get another 30 seconds of rest.

The results of the verification stage may be fed to stress classifier 110a and expectation stress detector 110b for analysis. The data from stress classifier 110 and expectation stress detector 110b may then be received by global stress analysis algorithm 110c, wherein the response for each question may be given an SR score, as detailed above. Because the significant event (e.g., key question) in each set is known, the SR scores can be compared to the expected results, so as to verify the accuracy of the SR scores determined by global stress analysis algorithm 110c.

In some embodiments, the present disclosure provides for an exemplary psycho-physiological test protocol which may measure psychophysiological response in a test subject, based, at least in part, on a series of test question segments interspersed by recovery periods. In some embodiments, each segment comprises test questions associated with a specific topic of interest. In some embodiments, a global stress signal baseline is established for the test subject at the beginning of the test protocol and/or separately for each segment. In some embodiments, a global stress signal is then estimated for the test subject during the various segments of the protocol, wherein an SR may be determined based on one or more of a reaction times of the test subject to test questions, global stress intensity during the test segment, and global stress signal recovery rate during the recovery periods.

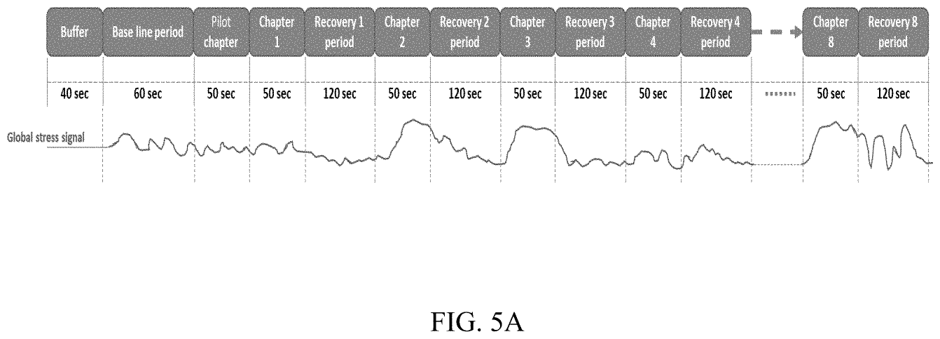

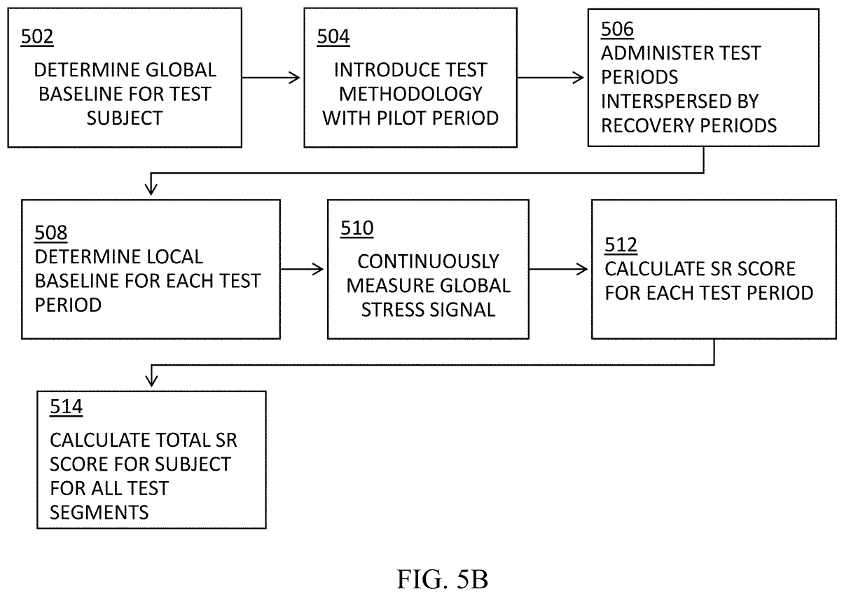

FIG. 5A is a schematic illustration of an exemplary test protocol according to an embodiment. In some embodiments, the test protocol comprises, e.g., a baseline period of between 30-180 seconds, and a pilot period of 50 seconds, followed by eight test periods of 30-80 seconds each, each followed by a recovery period of 30-120 seconds. In some embodiments, the test protocol may be configured to conduct a sequence of test periods followed by recovery stages, e.g., between 4 and 12 test segments, for example, 8 test segments. However, different, e.g., shorter or longer time periods may be used for all stages.

FIG. 5B is a flowchart detailing the functional steps in administering an exemplary psycho-physiological test protocol 500 to a participant, according to an embodiment. Protocol 500 can accommodate scenarios wherein stimulations of a subject comprise stimulation series segments.

Protocol 500 can also evaluate each stimulation series segment and base the SR score, at least in part on that evaluation. In some embodiments, the evaluation of stimulation series segments comprises stimulation series segment score. Such a score can be a numeric value, or a set of multiple numeric values in respect to recovery speed of the subject and/or the stress signal of the subject.

In some embodiments, at a stage 502, baseline physiological parameters of a test participant may be acquired, in a state of rest where the participant may not be exposed to any stimulations and/or only exposed to stimulations configured to induce a neutral emotional or cognitive state. For example, the participant may be exposed to one or more segments of undemanding cognitive tasks, to induce a neutral emotional state. In some embodiments, a global baseline stress signal may be determined of the test subject at stage 502. In some embodiments, the global stress baseline calculation may include:
(i) Global Baseline Signal Intensity Score: Average of the global stress signal during the baseline period.
(ii) Global Baseline Flat Score: Variance of the global stress signal during the baseline period.

At a stage 504, the subject may be exposed to a pilot segment. The pilot can take place prior to starting the stimulation series segments. In the pilot segment, the participant may be exposed to a sample test segment to acquaint the test subject with the test format. In some embodiments, the pilot segment is designed to demonstrate the structure of the stimulation series subset to the subject. In some cases, the pilot segment may take place for demonstration purposes, to ensure that the subject is familiar with the test structure, format, and methodology. In some embodiments, the pilot segment can continue for 50 seconds. In some embodiments, the pilot segment 504 can continue for between 30 and 60 seconds.

Following the pilot segment, at stage 506, a series of test segments are administered to the test subject. In some embodiments, each test segment comprises a plurality of relevant test questions on a single topic and/or subject of interest. In some embodiments, each test segment may also comprise, e.g., visual stimulations, auditory stimulations, and/or verbal stimulations.

In some embodiments, the stimulation can be a questionnaire comprising one or more sets of test questions, wherein each said set comprises an identical plurality of test questions arranged in a different order. In some embodiments, each said subset comprises relevant questions and non-relevant questions.

In some embodiments, at 508, a local stress signal baseline may be determined for the test subject before or at the beginning of each test segment. for example, local stress baseline may be determined based on a global stress signal measured during the last 1-5 seconds of the recovery period immediately preceding the test segment. In some embodiments, a segment-specific baseline may be calculated as an average between the global baseline calculated at 502, and the local baseline associated with the upcoming segment. In some embodiments, with respect to a test segment, the local baseline signal may be calculated as an average of the global stress signal during last 2 seconds before a test segment.

In some embodiments, a single test segment can continue for 50 second. In some embodiments, a single test segment can continue for between 30 and 60 seconds.

In some embodiments, a test segment may be formatted and/or structured based on one or more of the following guidelines:
(i) Each question may be presented on a screen with possible answers.
(ii) The test may include voice narration.
(iii) Each question may be associated with a relevant visualization displayed to the subject.
(iv) A maximum response time between questions may be set at, e.g., 4 seconds.
(v) A response timer countdown may be displayed to the subject.
(vi) At the pilot segment, the subject may be instructed with respect to response times and the significance of failing to respond on time.
(vii) The length of each subset may be set at a maximum of 50 seconds.
(viii) The recovery segment between the test segments is 120 second, and may include undemanding cognitive tasks.
(ix) Total test time may not exceed 25 minutes.
(x) Test questions may be formatted as multiple-choice questions.
(xi) Test subject physiological response may be enhanced by asking the test subject a follow-up question such as, “Are you sure?”, and/or “Would you agree to a polygraph test in connection with this answer?”.
(xii) Significant/relevant test questions may be interspersed with ‘non-relevant’ and/or simple questions.

In some embodiments, at stage 510 a global stress signal is continuously measured of the test subject throughout the test period. In some cases, the measuring and calculating of the stress signal may be based on a continuous measurement, wherein the stress signal is measured and calculated every predefined time interval. For example, the stress signal can be measured and calculated every one second, a half second, and the like. In some embodiments, during each of the stimulation segments, subject may be exposed to one or more test questions, each configured to induce one of the different categories of stress described above, including neutral emotional or cognitive stress, cognitive stress, positive emotional stress, negative emotional stress, and/or continuous expectation stress.

In some embodiments, at 512, a psychophysiological response and/or similar score may be calculated with respect to one, several, and/or all of the test segments separately, and/or for the entire test period. In some embodiments, a test segment score may be based, at least in part, on a combination of the following parameters:
(i) Reaction time: A calculation based on the average response time off the test subject to the test questions, i.e., elapsed time from the moment a question is presented to the subject and until the subject inputs an answer (e.g., by pressing a button).
(ii) Response Intensity: An area under the graph of the stress signal associated with a test segment.
(iii) Recovery Speed: A time to return to baseline stress values after a test segment, calculated as a combination of:
   a. Recovery Signal Intensity Score: Average stress signal intensity during a recovery period following a test segment; and
   b. Recovery Flat Score: A mean value of stress signal variance based on, e.g., overlapping time windows of, e.g., 5 seconds each.

In some embodiments segment score may be based, at least in part, on, e.g., a weighted average of response intensity and recovery speed. In some embodiments, reaction times during the pilot segment may be taken into account, e.g., to determine unusual and/or significant response times for a test subject.

In some embodiments, other mathematical operations, formulations or models may be used, for example, an integral over a period of time, calculation mean value in one or more stimulation segment, sum operation, and the like.

In some embodiments, the recovery speed can be calculated according to a recovery threshold, or recovery thresholds, wherein the recovery threshold can comprise a combination of the recovery signal intensity score and/or recovery flat score, and a threshold. Such a combination can be any mathematical operation such as sum, multiplication, and the like.

In some embodiments, SR may be determined based, at least in part, on whether the stress signal of the subject shows a recovery to baseline values. In some embodiments, the relevant baseline value may be the global baseline value and/or a relevant local baseline value with, e.g., a threshold value.

In some embodiments, a ‘normal’ response may be defined as a stress signal which may recover to baseline during the recovery period, regardless of intensity, such that:

Recovery Signal Intensity<Global Baseline Signal Intensity+Threshold

and

Recovery Flat Score<Global Baseline Flat+Threshold

In some embodiments, SR may be defined as a response intensity which does not subside to baseline during the recovery period, such that:

Recovery Signal Intensity>Global Baseline Signal Intensity+Threshold

OR

Recovery Flat Score>Global Baseline Flat+Threshold

In some embodiments, the threshold may be between 5-20% above baseline levels, e.g., 10%.

In some embodiments, responses of the subject, at the stimulations may be measured for determining whether the recovery speed indicates that the response is significant. In some embodiments, determining whether the response is a psychophysiological response may take into account recovery speed.

In some embodiments, the response level may be measured and compared with some recovery parameters as elaborated below. In some embodiments, the stress signal of a subject can be measured at the recovery segment.

In some embodiments, the recovery speed can be utilized to determine the stimulation series subset score. The recovery speed can be used in diverse computation options, integrals, deviations, and the like. In some embodiments, the stimulation series subset score can be determined by:

Response Intensity*Recovery Speed=stimulation series subset score

In some embodiments, at stage 514, a total SR score may be calculated for the subject, based on results of at least some, or all, of the test segments. In some embodiments, the total score may be based, at least in part, on a segment importance score, e.g., between 1 and 3, which may be assigned, e.g., by a designer and/or administrator of the test protocol.

In some embodiments, a maximum SR score for the entire test protocol may be calculated as:

(Maximum Total Score)=(Maximum Segment Score)*(Sum of all Segment Scores)(Importance Score of Segment i)

In some embodiments, a weighted segment risk score may be calculated as:

(Segment Risk Score)=(Segment Score)*(Segment Importance Score)

wherein the total SR score of the test protocol may be calculated as:

(Total Test Score)=100*sum(Segment i Score)/Maximum Total Score

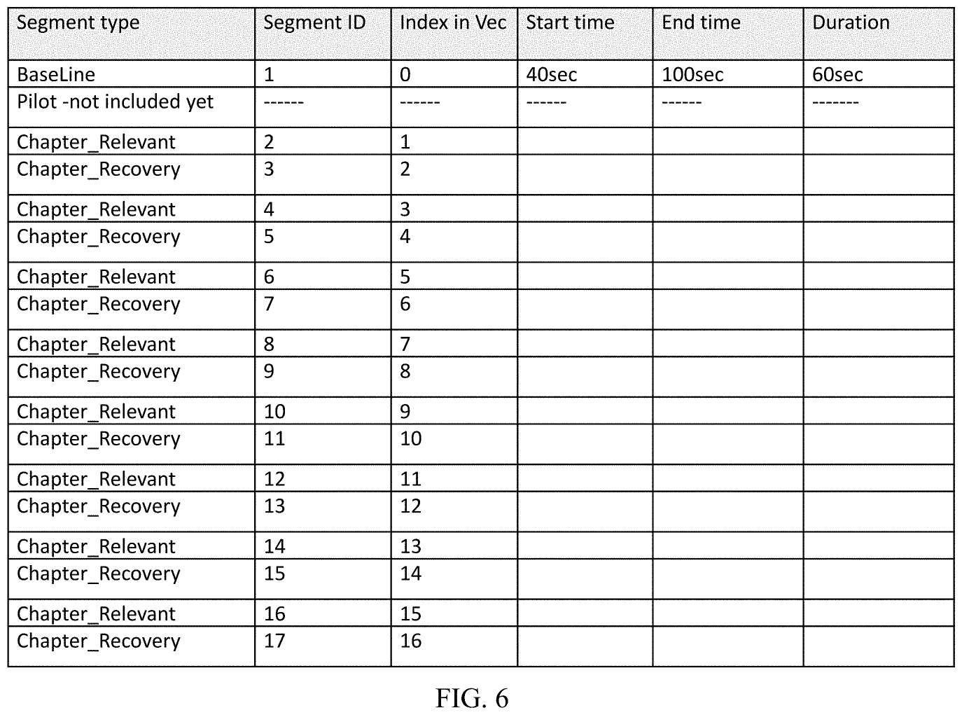

In some embodiments a stimulation series subset meta data, denoted herein as a subset metadata vector, as shown in FIG. 6, may be utilized to control data on over the stimulations in the subset. In some embodiments, this data can be used for calculating the time dependent factors such as some embodiments, recovery time, and the like.

FIG. 6 shows subset metadata vector wherein each object is a subset associated with a score, an average of each signal in that duration of time. In some embodiments, the subset metadata vector can be utilized as follows:

In some embodiments, an exemplary algorithm of the present disclosure provides for:
Calculating an offset of 20 seconds forward:

int calculateOffset(vector<subsetMetaData>&vectsubsetMetaData,double offset)
Smoothing all signals by moving average middle window —window size is, e.g., 10 seconds.
In the BaseLine subset, calculating the average in that subset for each of the stresses.
Calculation of average of the base line in smoothed global stress—set to be baselineAvg.
Calculation of flat signal is an overlapping window of 5 seconds, in every window the variance is calculated, inserted into a vector for average calculation. This is the flat!
In the Recovery segment need to calculate the average in that subset for each of the stresses.
Calculate the local baseline:the values is—last 5 second of the subset up until 3 second of the end of the subset. Duration is for 2 seconds at total−calc the avg of that two seconds. Don't need to calculate flat in the last recovery segment.
Calculate the flat average recovery and average recovery.
In the Relevant subset calculate the smoothed global stress DC by removing the baselineAvg.
In subset ID 2 (relevant subset) there is no recovery segment yet so calculation is different than others subset—Need to compute the integral of that signal, which will become the intensity of the relevant subset.
In subset ID that isn't 2 (relevant subset) the calculations can be: need to find the recovery subset that belong to the previse relevant subset and calc the avg between the local baseline recovery thereof and the baseline. The value and remove DC with that to the smoothed global stress and then calculate the integral of that signal, which becomes the intensity of the relevant subset.
Having all the intensities of relevant stimulation and the flat and average of all the recovery segments the following scoring is calculated.
Sort the intensities descending and score them from 0-7.
For each recovery segment take the biggest out of flat vs. avg into rec_score
Then for each subset multiply the Intensity and rec_score.
Scale the multiplication into scale of 0-5 by closest:
0->0
1->(1-7)
2->(8-14)
3->(15-21)
4->(22-28)
5->(29-35)

In some embodiments, an exemplary analysis flow may comprise:
(i) The final answer of the algorithm is based on two indicators:
(ii) Response intensity.
   a. Average response time in the subset—The time took the subject to answer the question once the question was finished.
   b. Recovery Time—Average and average of variance calculated from 5-second idle windows; and
(iii) The calculation of the intensity of the reaction in the subset is based on the area below the graph relative to the average of the initial (global) and local baseline.
(iv) If the duration of the stimulation is not the same it is required to calculate the relative area.
(v) Local baseline is considered 2 seconds before the start of the stimulation or at a specified time interval according to the content of the recovery period. In the initial stage the local baseline is calculated at 115-116 seconds from the start of the recovery period (2 seconds before the subject is required to mark the correct answer).
(vi) It is necessary to take into account the response delay in the system, ie to shift the relevant signals for analysis.
(vii) The use of response time of a subject to the question of the respondent's responses to the questions in the pilot section, or a rating in relation to other stimulations of the test.
(viii) Recovery level index—the speed of recovery, the return to the baseline of the mean intensity of the stress during the recovery period and the variance of the signal. If these two indices are below the baseline, the reaction is normal. The scale and the indices 10% above the baseline indicate that the subject did not recover.

In some embodiments, an exemplary test flow may comprise:
(i) Stimulations of buffer accumulation, initial basal construction, and recovery episodes do not require audio accompaniment.
(ii) Subset of questions and the pilot subset require voice guidance only with questions. The answers in these segments are unaccompanied by voice guidance.
   a. Accumulating primary buffer—subject demographic, personal, and/or attitudinal questions (e.g., educational attainment, age, preferences, workplace etc.).
(iii) Pilot Segment—general and/or personal questions (e.g., “Did you ever forget your cell phone at home and come back to pick it up?”, “I believe in cooperation at work”)
(iv) Exemplary test segments:
   a. Drug abuse
   b. Alcohol abuse
   c. Gambling habits
   d. Discretion
   e. Workplace theft
   f. Criminal and disciplinary history
   g. Corruption and bribery
(v) Exemplary recovery segments:
   a. Counting geometrical shapes/colors.

In some embodiments, the present disclosure provides for an automated analysis algorithm of a subject's psychophysiological responses to a test protocol, such as the test protocol detailed above with reference to FIGS. 5A-5B.

In some embodiments, the present algorithm is configured to detect and rate significant and/or suspicious reaction, e.g., SR, in test segments or chapter. In addition, in some embodiments, the present algorithm calculates a final score as a single indicative measure, which summarizes a subject's responses in the various test segments that are indicated as significant to the party administering the test.

In some embodiments, the analysis is based on detecting a subject's stress responses during answering questions in the relevant test segments. In some embodiments, rating response severity level may be performed in the segment, e.g., on a scale of 1-5, and is calculated in relation to subject-specific baseline. In some embodiments, the analysis is based, at least in part, on reaction levels in the subject during neutral and/or recovery periods in the test.

In some embodiments, the test protocol, as further detailed above with reference to FIGS. 5A-5B, is an ‘integrity’ test which is indicative of overall response to an entire test segment, rather to individual test questions.

As noted above, the test questions in each test segment typically relate to a single topic, without recovery times between questions. After each relevant segment, there is a recovery period in which the subject is engaged in simple cognitive tasks that allow the subject to recover, and serve the purpose of diverting the subject's attention for the previous segment. In some embodiments, the test protocol comprises a 60-second period at the beginning of the test to establish a baseline for the subject, in which the subject performs simple cognitive tasks (e.g., filling out a general questionnaire), to measure the level of reactivity of the subject in a neutral period without any triggers.

In some embodiments, the present algorithm receives as input a global stress signal measured in the course of administering the test protocol, as well as a vector of test events (type of segments, start and end times of segments), and a vector of segment importance weights as may be defined by a party administering the test.

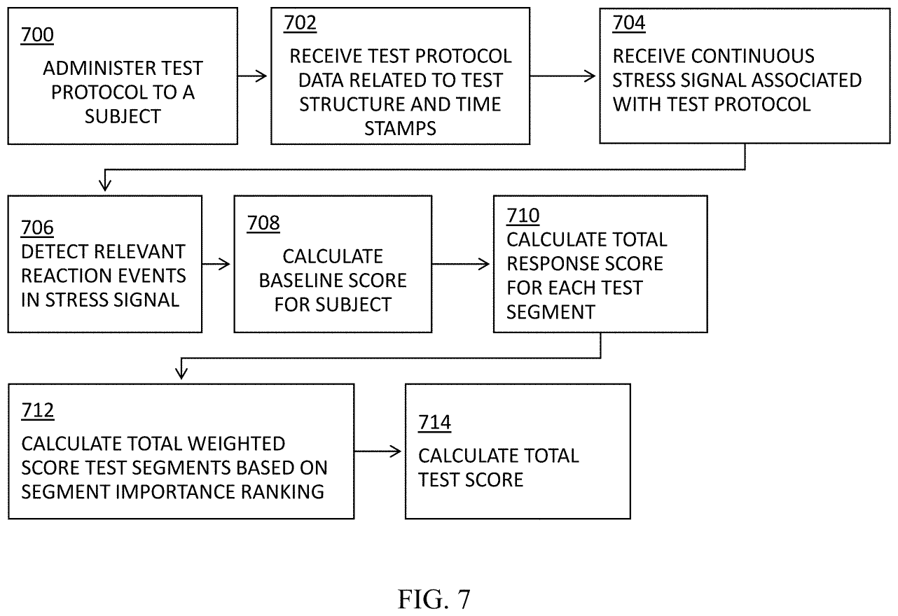

FIG. 7 is a flowchart of the functional steps in an algorithm of the present disclosure.

In some embodiments, at step 700, a test protocol, such as the test protocol described with reference to FIGS. 5A-5B may be administered to a subject.

In some embodiments, at step 702, the present algorithm receives as input data related to test protocol structure, including, but not limited to, number and time stamps of test segments, number and time stamps of test questions, and number and time stamps of recovery periods.

In some embodiments, at step 704, the present algorithm receives a global stress signal measured in the course of administering the test protocol.

In some embodiments, at step 706, the present algorithm analyzes the stress signal to detect one or more reaction sections in the stress signal, wherein a reaction section may be defined as an increase in an intensity value of the stress signal from a local minimum. In some embodiments, the present algorithm may be configured to determine at least some of reaction section start time, reaction section end time, and reaction section area under curve.

In some embodiments, at step 706, the present algorithm further calculates one or more response scores associated with the reaction sections determined at step 706.

In some embodiments, the present disclosure provides for one or more signal features which are most predictive and/or indicative of suspicious and/or significant reaction by a subject to the test protocol. In some embodiments, one of these feature may be a ‘reaction area,’ defined as the area under a reaction section in the stress signal curve, wherein a reaction section may be defined as an increase in an intensity value of the stress signal from a local minimum.

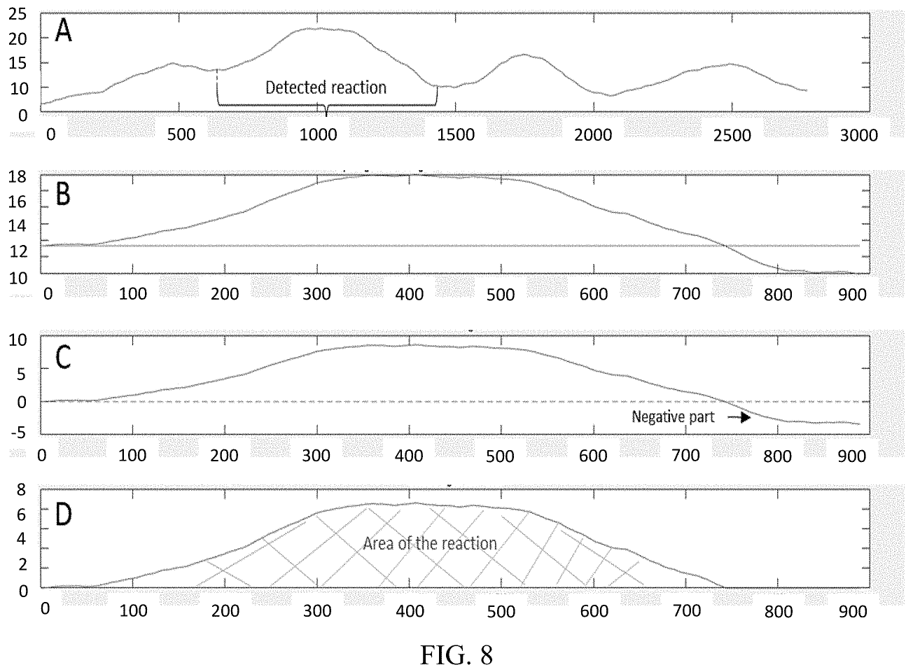

FIG. 8 is an illustration of reaction area calculation. Panel A shows a smoothed stress signal with a detected reaction on (as marked in parenthesis). Panel B shows an enlarged view of the reaction's stress signal and its baseline. Panel C shows the stress signal after baseline subtraction. Panel D shows the resulting signal after reset, from which the area is calculated.

In some embodiments, this feature expresses the overall intensity which considers both the duration of a detected reaction and its intensity (including the recovery phase) regardless of the reaction type (global or secondary). In some embodiments, the calculation of the reaction area may be performed as follows:

For each Detected Reaction do

InputSignal=stress signal(Reaction.startInd:Reactions.endInd);

DC_Baseline=global_signal(Reactions.start_Ind);

(% local baseline at the start reaction)

Reaction.Area=AreaCalculation(InputSignal,DC_Baseline);

End For

In some embodiments, the ‘AreaCalculation’ may be based on a trapezoid method. The function subtracts the DC form the original signal and resets the negative values in the resulted signal before area calculation. Thus, the reaction area is calculated only from the positive part of the resulted signal.

In some embodiments, the present disclosure may provide for calculating a reaction score, which expresses the intensity of the reaction in combination with the duration of the reaction (including the recovery phase), taking into account the type of the reaction (global or secondary reaction). In some embodiments, reaction score may be calculated as:

Reaction Score=Duration Ratio*Relative Max Amplitude
Duration Ratio: How large or small the duration of a reaction is relative to a normal reaction duration associated with each type of reaction. Accordingly,

Duration Ratio=Duration of the Reaction/Normal Duration

Relative Max Amplitude: Expresses the intensity of the reaction relative to the local baseline from which the reaction started. This parameter is calculated as follows:

Relative Max Amplitude=Max magnitude−stress value at the beginning of the reaction.

### Relevant Reaction Detection

In some embodiments, with continued reference to step 706 in FIG. 7, the present algorithm is configured to detect reactions sections in the stress signal, based, at least in part, on a measure of one or more reaction areas in the stress signal.

In some embodiments, the present algorithm is further configured to associate the one or more reaction areas with start and/or end times of test segments, e.g., relevant test segments. In some embodiments, test segments start times may be adjust in connection with such associations, e.g., by shifting start times a specified time period, e.g., 4 seconds.

In some embodiments, for each relevant test segment, all associated reaction area measured by the present algorithm may be aggregated. Accordingly, in some embodiments, within a relevant test segment, total reaction area of the segment is the sum of all reaction areas of all relevant reactions within the segment.

### Baseline Score

In some embodiments, at step 708, the present algorithm may be configured to calculate a baseline score which reflects a subject's baseline reactivity, i.e., the normal pattern if subject reactions during relevant and non-relevant events (which may include the baseline period and the recovery periods of the test protocol). In some embodiments, a subject's baseline score may combine two characteristics, one indicating the baseline reaction intensity and the other indicating the level of scores variability throughout the test.

In some embodiments, a baseline score may be calculated using the reactions area calculation, e.g., of all detected reactions during a test protocol. In some embodiments, the calculation of the Baseline score as follows:

Baseline Reaction Intensity=Average(Reactions.Area)

Reaction Variability=Standard deviation(Reactions. Area)

Baseline Score=Baseline Reaction Intensity+Reaction Variability

### Segment Total Response Score Calculation

In some embodiments, at step 710, the present algorithm may be configured to calculate a total response score for a test segment, equal to the final segment score that expresses the severity of the detected psychophysiological response during the relevant segment. This score is calculated based on total reaction area of the detected relevant reactions during the test segment and the baseline score. The response severity in a relevant test segment is basically the rating of the distance between the reaction area value and the baseline score. The greater the distance, the more severe the reaction to the test segment.

In some embodiments, test segment total response score (e.g., severity level) is rated on a scale of 1 to 5 according to predefined thresholds. In some embodiments, such rating determines how large/significant the response is. The thresholds and ratings are listed in table 1 below.

TABLE 1
Segment Total Response Score calculation

| | Test Segment Total |
|---|---|
| Threshold | Response score |
| Reaction Area < Baseline score * 0.5 | 1 |
| Baseline score * 0.5 ≤ Reaction Area < | 2 |
| 0.9 * Baseline score | |
| Baseline score * 0.9 ≤ Reaction Area < | 3 |
| Baseline score * 1.4 | |
| Baseline score * 1.4 ≤ Reaction Area < | 4 |
| Baseline score * 1.9 | |
| Reaction Area ≥ Baseline score * 1.9 | 5 |

The thresholds listed in table 1 were empirically determined based on a training set. In some embodiments, additional and/or other thresholds may be used.

Total Weighted Score of Relevant segments

In some embodiments, at step 712, the present algorithm may be configured to calculate a total weighted score for a relevant test segment, wherein the total relevant score combines the actual physiological response during the test segment (e.g., the total segment score) with an importance level associated with the test segment, as may be user-indicated or assigned.

In some embodiments, test segment importance weight may be assigned by the party administering and/or designing the test protocol, e.g., on a scale of 1-3 (e.g., 1—low importance, 3—high importance). In some embodiments, the total weighted score expresses how much a subject's response to a particular test segment is considered problematic in view of the party administering the test. That is, even if the score of a physiological response to a particular segment is high (e.g., 5), this segment could get a low total weighted score if the segment is rated with relatively lower importance.

In some embodiments, the input of this step is the vector of segment total response score and a vector of segment importance weights. The output is a vector of Total weighted scores all relevant segment in 0-100 scale. The score is calculated as follows:

$$Max segment score = 5 // max possible score of segment Total Response$$

$$Max importance weight = 3$$

$$Segment Weighted Score = 100 \times \frac{{Segment}_{i} Importance Weight \times {Segment}_{i} Total Response Score}{Max Segment Score \times times Max Importance Weight}$$

### Final Test Score

In some embodiments, at step 714, the present algorithm may be configured to calculate a total test score, based on the total test segment scores. In some embodiments, the weighted segment score may be used, while other embodiments, the total score in unweighted.

In some embodiments, total test score calculation depends on the severity of total segment scores, and consists of the maximal scores of each segment, with the addition of an appropriate bias according to maximal total score severity. In some embodiments, test segment total score severity may be defined as shown in table 1 above, e.g., scores of 1-2 indicate insignificant response, 3 indicate medium response, 4 indicates severe response, and 5 indicates very severe response. Accordingly, each type of test segment total score is weighted according to its severity in the final score. The bias for each type of total score severity has been empirically defined so that the final score will optimally reflect the results of the entire test. The weight of each type of total score in the final score is calculated as follows:

$$severity i = 1 , 2 , 3 , 4 , 5 \\ Max umber of score repetition = 8 \\ Weight in final score ( severity i ) = \frac{Empiric weight ( severity i )}{Max numberofscorerepetition}$$

Table 2 lists the severity empiric weighs of segment total scores:

TABLE 2
Empiric Severity Weighs of Segment Total Scores

| Segment Total Score (severity i) | Empiric Severity weigh |
|---|---|
| 1 | 0% |
| 2 | 10% |
| 3 | 15% |
| 4 | 20% |
| 5 | 55% |

The results of weighs in final score calculation and the Bias are listed in table 3 below:

TABLE 3
Final Score Components

| Segment Total Score | | |
|---|---|---|
| (severity i) | Weigh in final score | Bias |
| 1 | 0 | 0 |
| 2 | 1.25 | 0 |
| 3 | 1.875 | 10 |
| 4 | 2.5 | 25 |
| 5 | 6.875 | 45 |

Following are the Final Risk Score and Final Test score formulas:

Final Risk Score=Bias+number of MaxScore repetition×Weight in final score

Final Test Score=100−Final Risk Score

Note that the scale of Final Risk Score and Final Test Score is 0-100.

The pseudo-code of Final Risk Score and Final Test Score calculation as follows:

```
// find the maximal Segment Total Score of the test
maxScoreIn WholeExam = max (Segment Total Score vector);
// rank/count repetition of Segment Total Scores
For i=1:numOfSegments do
  Segment Total Scores (i) ==1 → rank(1)=+1;
  Segment Total Scores (i) ==2 → rank(2)=+1;
  Segment Total Scores (i) ==3 → rank(3)=+1;
  Segment Total Scores (i) ==4 → rank(4)=+1;
  Segment Total Scores (i) ==5 → rank(5)=+1;
End for
// Final Risk Score calculation (based on parameters listed in Table 3)
If maxScoreInWholeExam == 1 then
Final Risk Score = 0 + rank(1) * 0;
End if
If maxScoreInWholeExam == 2 then
Final Risk Score = 0 + rank(2) * 1.25;
End if
If maxScoreInWholeExam == 3 then
Final Risk Score = 0 + rank(3) * 1.875;
End if
If maxScoreInWholeExam == 4 then
Final Risk Score = 0 + rank(4) * 2.5;
End if
If maxScoreInWholeExam == 5 then
Final Risk Score = 0 + rank(5) * 6.875;
End if
// Final Test Score calculation
Final Test Score = 100 − Final Risk Score;
Return (Final Risk Score, Final Test Score);
```

### Segment Recommendation

In some embodiments, with respect to each segment, the present algorithm determines if the responses that were detected in the segment are within the normal range, to highlight test abnormalities in the test analysis. For example, if answers of a subject on a particular segment are within a normal range, but the analysis finds abnormalities in the test segment (e.g., high severity scores of 3-5), this may serve as an indication that the credibility of the subject in this segment is questionable.

Measures of Emotional and Cognitive stress

In some embodiments, the present algorithm may be configured to measure emotional and cognitive intensity during relevant segments of a test protocol, to provide additional insights to the test results. Accordingly, the present algorithm calculates average intensity of the stress signal during a test segment.

### Algorithm Output

In some embodiments, the present algorithm may be configured to output at least some of the following data:
(i) Total segment response score,
(ii) Segment weighted response score,
(iii) Total test score,
(iv) Test abnormality indication,
(v) Test emotional stress average intensity, and
(vi) Test cognitive stress average intensity.

### Test Result

In some embodiments, the present algorithm may be configured to output a total test result, which is a literal interpretation of the final test score. The calculation of the test result may be based on the final test score according to the ranges listed in table 4:

TABLE 4
Test Recommendation Description

| | Range of Final | |
|---|---|---|
| Recommendation | Test Score | Description |
| Recommended | 90-100 | All segments has insignificant |
| | | response |
| Clarification | 75-89 | At least 1 segment with Segment |
| Required | | total score 3 |
| | | (medium intensity) |
| Marginal | 55-74 | At least 1 segment with Segment |
| | | total score 4 |
| | | (high intensity) |
| Not | 0-54 | At least 1 segment with Segment |
| Recommended | | total score 5 |
| | | (very high intensity) |
| Inconclusive | Not enough | Technical issues |
| | data | |

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A system comprising:
at least one hardware processor; and
a non-transitory computer-readable storage medium having stored thereon program instructions, the program instructions executable by the at least one hardware processor to:
receive, as input, physiological parameters data measured in a human subject in response to an administered test question protocol comprising (a) a plurality of test question segments, each comprising at least one test question, and (b) a recovery period following each of said test question segments,
determine a stress signal associated with said test question protocol, based, at least in part, on one or more states of stress detected in said physiological parameters data,
temporally associate values of said stress signal with said plurality of test question segments and said recovery periods, and
calculate, for at least some of said test question segments, a segment psychophysiological response score associated with said responses by said subject, based on an analysis of said temporally associated values of said stress signal.

2. (canceled)

3. (canceled)

4. The system of claim 1, wherein said analysis comprises calculating at least one of:
(i) a test question protocol stress signal global baseline associated with said subject, based, at least in part, on said values of said stress signal during said baseline period; and
(ii) with respect to each test question segment, a stress signal segment baseline, based, at least in part, on said global baseline and a value of said stress signal during a said recovery period immediately preceding said test question segment.

5. The system of claim 1, wherein said analysis comprises, with respect to a test question segment of said test question segments, calculating at least one of:
(i) reaction times associated with each of said responses to each of said test questions;
(ii) an intensity value of said stress signal associated with said test question segment, relative to said test question segment baseline; and
(iii) an intensity and variability values of said stress signal during a said recovery period immediately following said test question segment, relative to said global baseline, and
wherein said segment psychophysiological response score is based, at least in part, on said calculating.

6. (canceled)

7. The system of claim 1, wherein said analysis comprises detecting one or more reaction sections in said stress signal, based, at least in part, on an increase in said value of said stress signal relative to a local minimum, and wherein said analysis further comprises calculating an area under a curve associated with each of said reaction sections.

8. (canceled)

9. The system of claim 7, wherein said analysis further comprises calculating a test question protocol stress signal global baseline associated with said subject, based, at least in part, on an (i) average of all of said areas under said curve associated with each of said reaction sections, and (ii) a variability of all of said areas under said curve associated with each of said reaction.

10. The system of claim 9, wherein said segment psychophysiological response score is based, at least in part, on a sum of all of said areas under said curve associated with each of said reaction sections, associated with said respective test question segment, relative to said global baseline.

11. The system of claim 9, wherein said segment psychophysiological response score is based, at least in part, on a reaction score associated with said test question segment, equal to a duration of said reaction section relative to a standard reaction duration, multiplied by an intensity value of said stress signal during said reaction section.

12. The system of claim 1, wherein said program instructions are further executable to calculate a test question protocol psychophysiological response score, based, at least in part, on a weighted sum of all of said segment psychophysiological response scores, weighting is based on one of: score severity and test question segment importance ranking.

13. (canceled)

14. The system of claim 1, wherein said states of stress are selected from the group consisting of: neutral stress, cognitive stress, positive emotional stress, and negative emotional stress, wherein said stress signal is calculated, at least in part, by combining at least one of said detected cognitive stress, positive emotional stress, and negative emotional stress.

15. (canceled)

16. The system of claim 1, wherein said physiological parameters data are acquired using one or more of: an imaging device, an infrared (IR) sensor; a hyperspectral imaging device; a skin surface temperature sensor; a skin conductance sensor; a respiration sensor; a peripheral capillary oxygen saturation (SpO2) sensor; an electrocardiograph (ECG) sensor; a blood volume pulse (BVP) sensor; a heart rate sensor; a surface electromyography (EMG) sensor; an electroencephalograph (EEG) acquisition sensor; a joint bend sensor; and a muscle activity sensor.

17. A method comprising:
receiving, as input, physiological parameters data measured in a human subject in response to an administered test question protocol comprising (a) a plurality of test question segments, each comprising at least one test question, and (b) a recovery period following each of said test question segments,
determining a stress signal associated with said test question protocol, based, at least in part, on one or more states of stress detected in said physiological parameters data,
temporally associating values of said stress signal with said plurality of test question segments and said recovery periods, and
calculating, for at least some of said test question segments, a segment psychophysiological response score associated with said responses by said subject, based on an analysis of said temporally associated values of said stress signal.

18. (canceled)

19. (canceled)

20. The method of claim 17, wherein said analysis comprises calculating at least one of:
(i) a test question protocol stress signal global baseline associated with said subject, based, at least in part, on said values of said stress signal during said baseline period; and
(ii) with respect to each test question segment, a stress signal segment baseline, based, at least in part, on said global baseline and a value of said stress signal during a said recovery period immediately preceding said test question segment.

21. The method of claim 17, wherein said analysis comprises, with respect to a test question segment of said test question segments, calculating at least one of:
(i) reaction times associated with each of said responses to each of said test questions;
(ii) an intensity value of said stress signal associated with said test question segment, relative to said test question segment baseline; and
(iii) an intensity and variability values of said stress signal during a said recovery period immediately following said test question segment, relative to said global baseline.

22. The method of claim 21, wherein said segment psychophysiological response score is based, at least in part, on said calculating, wherein said analysis comprises detecting one or more reaction sections in said stress signal, based, at least in part, on an increase in said value of said stress signal relative to a local minimum, and wherein said analysis further comprises calculating an area under a curve associated with each of said reaction sections.

23. (canceled)

24. (canceled)

25. The method of claim 22, wherein said analysis further comprises calculating a test question protocol stress signal global baseline associated with said subject, based, at least in part, on an (i) average of all of said areas under said curve associated with each of said reaction sections, and (ii) a variability of all of said areas under said curve associated with each of said reaction.

26. The method of claim 25, wherein said segment psychophysiological response score is based, at least in part, on a sum of all of said areas under said curve associated with each of said reaction sections, associated with said respective test question segment, relative to said global baseline.

27. The method of claim 25, wherein said segment psychophysiological response score is based, at least in part, on a reaction score associated with said test question segment, equal to a duration of said reaction section relative to a standard reaction duration, multiplied by an intensity value of said stress signal during said reaction section.

28. The method of claim 17, further comprising calculating a test question protocol psychophysiological response score, based, at least in part, on a weighted sum of all of said segment psychophysiological response scores, and wherein the weighting is based on one of: score severity and test question segment importance ranking.

29. (canceled)

30. The method of claim 17, wherein said states of stress are selected from the group consisting of: neutral stress, cognitive stress, positive emotional stress, and negative emotional stress, and wherein said stress signal is calculated, at least in part, by combining at least one of said detected cognitive stress, positive emotional stress, and negative emotional stress.

31. (canceled)

32. The method of claim 17, wherein said physiological parameters data are acquired using one or more of: an imaging device, an infrared (IR) sensor; a hyperspectral imaging device; a skin surface temperature sensor; a skin conductance sensor; a respiration sensor; a peripheral capillary oxygen saturation (SpO2) sensor; an electrocardiograph (ECG) sensor; a blood volume pulse (BVP) sensor; a heart rate sensor; a surface electromyography (EMG) sensor; an electroencephalograph (EEG) acquisition sensor; a joint bend sensor; and a muscle activity sensor.

33.-48. (canceled)
